(19) United States
(12) Patent Application Publication
(10) Pub. No.: US 2024/0277473 A1
(43) Pub. Date: Aug. 22, 2024

(54) RADIALLY EXPANDABLE PROSTHETIC DEVICE

(71) Applicant: EDWARDS LIFESCIENCES CORPORATION, Irvine, CA (US)

(72) Inventors: Michael Bukin, Pardes Hanna (IL); Nikolai Gurovich, Hadera (IL); Anatoly Dvorsky, Haifa (IL); Eran Grosu, Kfar-Saba (IL); Noam Nir, Pardes-Hanna (IL); Joseph Mordechai Leichner, Netanya (IL)

(21) Appl. No.: 18/645,284

(22) Filed: Apr. 24, 2024

Related U.S. Application Data
(63) Continuation of application No. PCT/US2022/049543, filed on Nov. 10, 2022.
(60) Provisional application No. 63/420,013, filed on Oct. 27, 2022.
(60) Provisional application No. 63/277,959, filed on Nov. 10, 2021.

(51) Int. Cl. A61F 2/24

(52) U.S. Cl. CPC A61F 2/243; A61F 2/2418; A61F 2250/001

(57) ABSTRACT

A prosthetic device, such as a prosthetic heart valve, can include a radially compressible and expandable frame and a screw actuator that is coupled to the frame and configured to be rotated to radially expand the frame from a radially compressed state to a first radially expanded state. The frame includes an over-expansion structure that is configured to allow relative sliding movement between the screw actuator and the frame in a longitudinal direction to permit the frame to radially expand from the first radially expanded state to a second radially expanded state when an external expansion force applied to the prosthetic heart valve exceeds a predetermined force.

### CROSS-REFERENCE TO RELATED APPLICATIONS

The present application is a continuation of PCT Application No. PCT/US2022/049543, filed Nov. 10, 2022, which claims the benefit of U.S. Provisional Application No. 63/420,013, filed Oct. 27, 2022, and U.S. Provisional Application No. 63/277,959, filed Nov. 10, 2021, all of which applications are incorporated herein by reference.

### FIELD

The present disclosure relates to implantable, radially expandable prosthetic devices, such as prosthetic heart valves, and to methods, assemblies, and apparatuses for delivering, expanding, implanting, and deploying such prosthetic devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various fully functional working diameters. Some mechanically expandable prosthetic heart valves can also be compressed after an initial expansion (for example, for repositioning and/or retrieval).

Despite the recent advancements in percutaneous valve technology, there remains a need for improved transcatheter prosthetic devices. Specifically, there remains a need for mechanically expandable prosthetic devices that can be over-expanded, such as to accommodate another prosthetic device. For example, when a prosthetic heart valve reaches the end of its operational life, rather than removing the expired prosthetic heart valve and replacing it with a new prosthetic heart valve, it may be desirable to place the new prosthetic heart valve within the existing prosthetic heart valve. As another example, a prosthetic heart valve may need to be further expanded during its operational life to maintain a seal with the native anatomy, such as if a patient's native heart valve grows/enlarges during the operational life of the prosthetic heart valve.

### SUMMARY

The present disclosure relates to implantable, mechanically expandable prosthetic devices, such as prosthetic heart valves, and to methods, assemblies, and apparatuses for delivering, expanding, implanting, and deploying such prosthetic devices.

A prosthetic device, such as a prosthetic heart valve, can include a radially expandable and compressible frame and a threaded rod or screw actuator that is coupled to the frame and configured to be rotated to radially expand the frame from a radially compressed state to a radially expanded state.

In some examples, the frame comprises an over-expansion structure that is configured to allow relative sliding movement between the screw actuator and the frame in a longitudinal direction to permit the frame to radially expand from a first radially expanded state to a second radially expanded state when an external expansion force applied to the prosthetic heart valve exceeds a predetermined force.

In some examples, the frame comprises a plurality of pairs of first and second axially extending posts, wherein the first and second posts of each pair are axially moveable relative to each other when the frame is radially expanded, wherein the screw actuator extends through a bore of the first post of a selected pair of posts and is coupled to the second post of the selected pair.

In some examples, the screw actuator comprises a stopper that is positioned between the first post and the second post of the selected pair, and wherein the first post comprises the over-expansion structure, which defines a cavity that is configured to selectively permit the stopper to move within the cavity, thereby allowing the frame to radially expand to the second radially expanded state when an external expansion force applied to the prosthetic heart valve exceeds a predetermined force.

In some examples, the over-expansion structure comprises flexible arms that extend axially from an end of the first post of the selected pair of posts.

In some examples, the screw actuator is configured to slide within the bore of the first post of the selected pair of posts without rotating when the external expansion force applied to the frame exceeds the predetermined force.

In some examples, the second post of the selected pair of posts comprises a bore through which the screw actuator extends, wherein the bore of the second post comprises threads that engage with mating threads on the screw actuator to couple the screw actuator to the second post and that prevents radial compression of the prosthetic heart valve unless the screw actuator is rotated.

In some examples, the second post of the selected pair of posts houses a threaded nut that engages mating threads on the screw actuator to couple the screw actuator to the second post and that prevents radial compression of the prosthetic heart valve unless the screw actuator is rotated.

In some examples, the over-expansion structure comprises a slot form in the second post of the selected pair of posts, at least one protrusion extending from an inner wall of the slot and defining first and second sections of the slot, and a nut disposed in the slot, wherein the screw actuator has external threads that engage internal threads of the slot, wherein the nut is disposed in and slidably within the slot between the first and second sections of the slot, wherein the protrusion retains the nut in the first slot section when the rod is rotated to expand the frame from the radially compressed state to the first expanded state and permits the nut to slide into the second slot section to allow the frame to further expand to the second expanded state when the external expansion force applied to the frame exceeds the predetermined force.

In some examples, the prosthetic device comprises a valvular structure comprising a plurality of leaflets supported inside of the frame, wherein the leaflets are configured to regulate the flow of blood through the frame in one direction.

In some examples, a prosthetic device comprises a frame. The frame comprises axially extending first and second posts that are axially movable relative to one another to permit the prosthetic device to radially expand and compress, wherein the first post comprises an inner bore. A threaded rod extends through the inner bore of the first post and is coupled to the second post, wherein the rod is configured to radially expand and radially compress the frame only when rotated but is otherwise configured to prevent radial expansion and compression of the prosthetic device. One of the first post and the second post further comprises an over-expansion structure that is configured to selectively permit the first and second posts to move axially closer together, when an external expansion force applied to the frame exceeds a predetermined threshold force, to allow the frame to radially expand even when the rod is not rotated.

In some examples, a prosthetic heart valve comprises a radially compressible and expandable frame and a screw actuator that is coupled to the frame and configured to be rotated to radially expand the frame from a radially compressed state to a first radially expanded state. The frame comprises an over-expansion structure that is configured to allow relative sliding movement between the screw actuator and the frame in a longitudinal direction to permit the frame to radially expand from the first radially expanded state to a second radially expanded state when an external expansion force applied to the prosthetic heart valve exceeds a predetermined force.

In some examples, a prosthetic device comprises a frame, the frame comprising an expansion and locking mechanism comprising an axially extending post comprising a proximal member and a distal member that are axially movable relative to one another to permit the prosthetic device to radially expand and compress. The expansion and locking mechanism further comprises a rod configured to radially expand and/or radially compress the frame when actuated but that is configured to prevent radial expansion and radial compression of the prosthetic device when not actuated. The rod comprises a head portion that is configured to provide an axially directed force in a first direction to the proximal member to radially expand the prosthetic device and a stopper that is configured to provide an axially directed force in a second direction to the proximal member to radially compress the prosthetic device. The proximal member of the post further comprises an over-expansion structure at a distal end of the proximal member that is configured to selectively permit the frame to radially expand even when the rod is not actuated.

In some examples, a prosthetic valve comprises a plurality of first posts extending between a proximal end and a distal end of the frame, each of the plurality of first posts comprising a proximal member and a distal member that are axially movable relative to one another to permit the prosthetic device to radially expand and compress, wherein the proximal member and/or the distal member comprise an inner bore. The prosthetic valve further comprises at least one rod extending through the inner bore of the proximal member of a selected one of the first posts and coupled to the distal member of the selected first post, wherein the rod is configured to radially expand and radially compress the frame only when rotated but is otherwise configured to prevent radial expansion and/or compression of the prosthetic device. The proximal member of the selected first post further comprises an over-expansion structure at a distal end of the proximal member that is configured to selectively permit the proximal member to move axially relative to the rod, towards the distal member, to allow the frame to radially expand even when the rod is not rotated. A plurality of second posts extend between a proximal end and a distal end of the frame, one or more of the plurality of second posts comprising a commissure support structure. A plurality of struts extend between and connect the plurality of first posts and the plurality of second posts. A valvular structure comprises leaflets, wherein tabs of adjacent leaflets are secured to the commissure support structures to couple the valvular structure to the frame.

In some examples, an assembly comprises a prosthetic device comprising a frame, the frame comprising one or more expansion and locking mechanisms. Each of the expansion and locking mechanisms comprises a post comprising a proximal member and a distal member that are axially movable relative to one another to permit the prosthetic device to radially expand and compress, and a rod configured to radially expand and/or radially compress the frame when actuated but that is configured to prevent radial expansion and/or radial compression of the prosthetic device when not actuated. The rod comprises a head portion that is configured to provide an axial compressive force to the proximal member to radially expand the prosthetic device and a stopper that is configured to provide an axial expansive force to the proximal member to radially compress the prosthetic device. The proximal member of the post further comprises an over-expansion structure at a distal end of the proximal member that is configured to selectively permit the frame to radially expand even when the rod is not actuated. The assembly further comprises a delivery apparatus comprising one or more actuator assemblies configured to radially expand and/or compress the prosthetic device, each of the one or more actuator assemblies comprising a sleeve and a driver releasably coupled to the rod of one of the one or more expansion and locking mechanisms, each driver configured to rotate the rod to which it is coupled to radially expand and/or compress the prosthetic device. The delivery apparatus further comprises a handle comprising one or more control mechanisms, at least one of the one or more control mechanisms configured to be actuated to rotate the driver of the one or more actuator assemblies and the rod of the prosthetic device to radially expand and/or compress the prosthetic device.

In some examples, a method of radially expanding an implanted prosthetic heart valve is provided. The prosthetic heart valve comprises a frame and at least one screw actuator, wherein the prosthetic heart valve is in a radially expanded state within a native heart valve. The method comprises radially expanding the prosthetic heart valve from the radially expanded state to a further radially expanded state without actuating the screw actuator of the prosthetic heart valve by applying an external expansion force to the prosthetic heart valve.

The above method(s) can be performed on a living animal or on a simulation, such as on a cadaver, cadaver heart, anthropomorphic ghost, simulator (e.g., with body parts, heart, tissue, etc. being simulated).

In some examples, a prosthetic device, such as a prosthetic heart valve, comprises one or more of the components recited in Examples 1-61 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic device, according to one example.

FIG. 1B is a side elevation view of a frame of the prosthetic device of FIG. 1A.

FIG. 2 is a side elevation view of a delivery apparatus for a prosthetic device, according to one example.

FIG. 3 is a perspective view of a portion of an actuator of the prosthetic device of FIGS. 1A-1B and an actuator assembly of a delivery apparatus, according to one example.

FIG. 4 is a perspective view of the actuator and actuator assembly of FIG. 3 with the actuator assembly physically coupled to the actuator.

FIG. 5A is a side elevation view of a proximal end portion of a frame of a prosthetic device, according to one example, in which the frame is in a radially expanded state.

FIG. 5B is a side elevation view of the proximal end portion of the frame of FIG. 5A in which the frame is in an even more radially expanded state than in FIG. 5A.

FIG. 6A is a side elevation view of a proximal end portion of a frame of a prosthetic device, according to another example, in which the frame is in a radially expanded state.

FIG. 6B is a side elevation view of the proximal end portion of the frame of FIG. 6A in which the frame is in an even more radially expanded state than in FIG. 6A.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

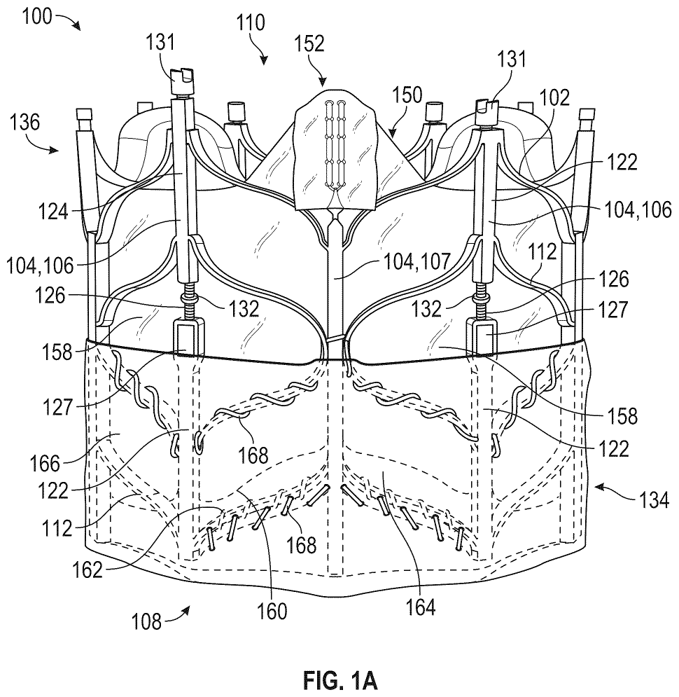

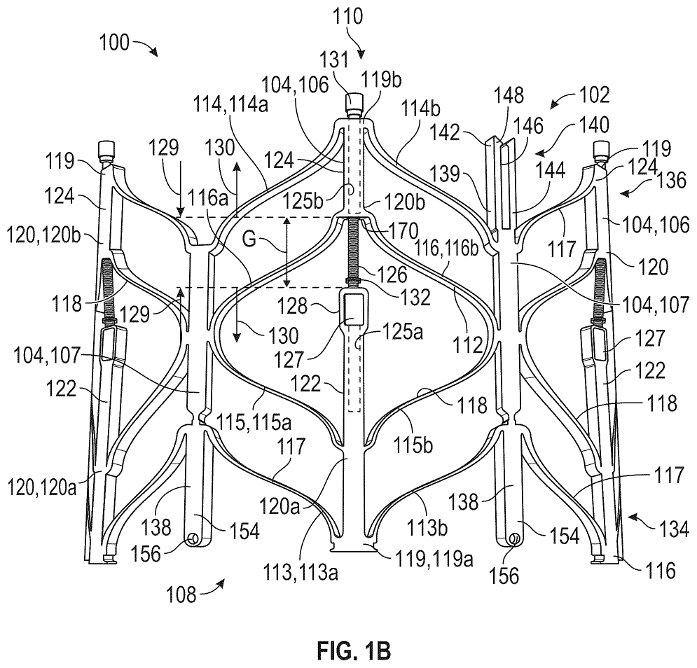

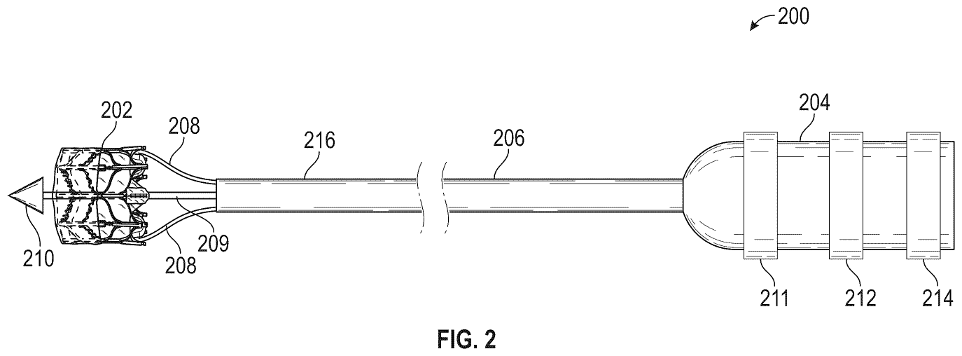

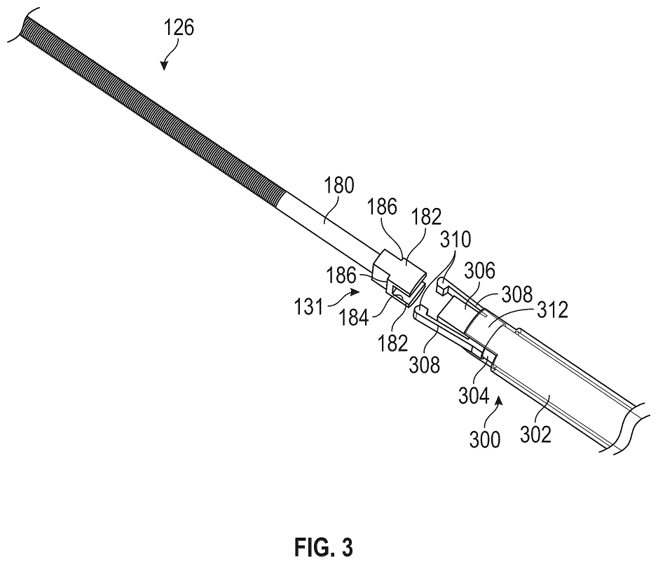

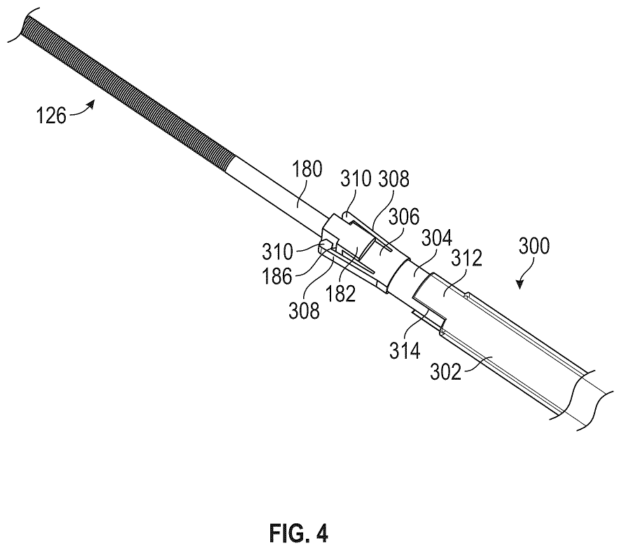

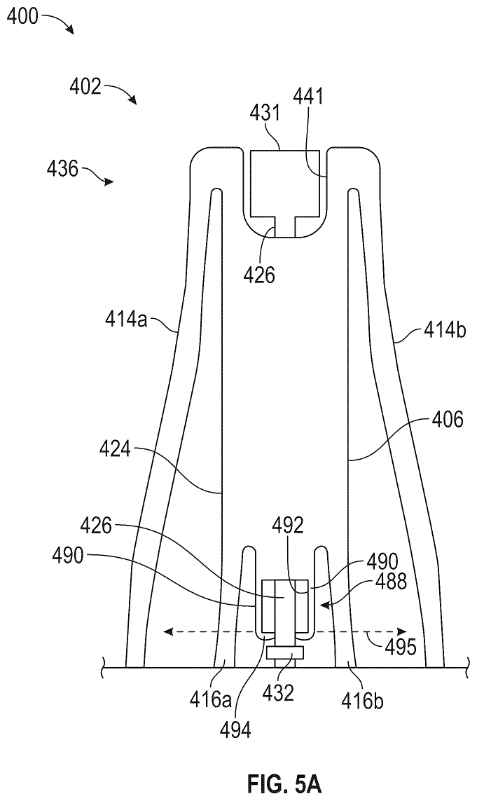

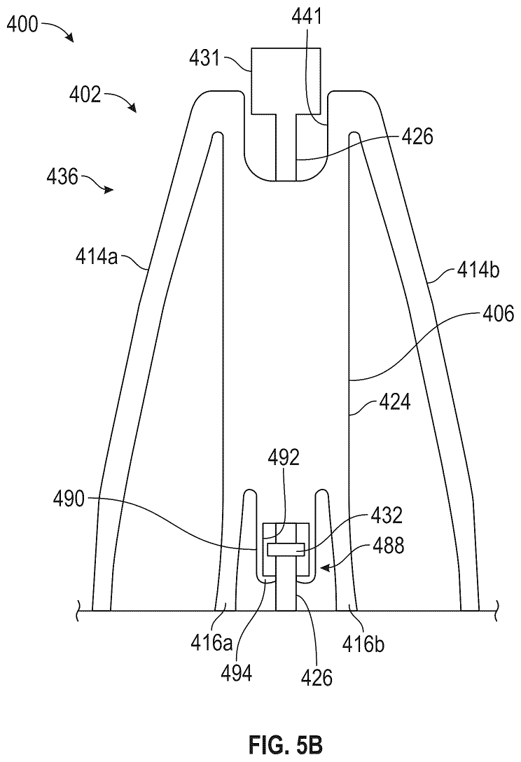

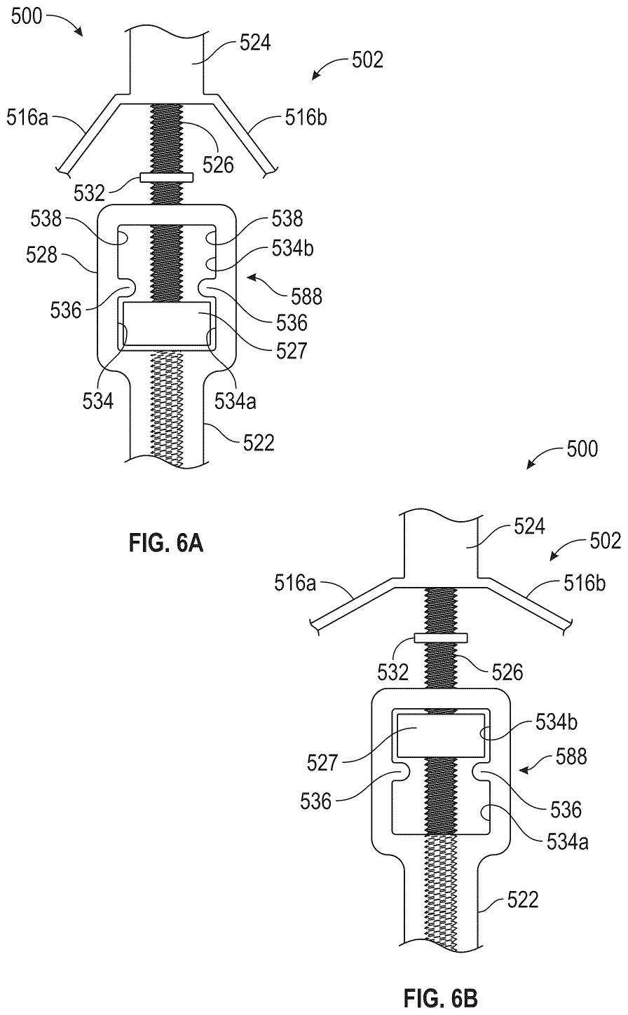

All features described herein are independent of one another and, except where structurally impossible, can be used in combination with any other feature described herein. For example, a delivery apparatus 200 as shown in FIG. 2 and/or an actuator assembly 300 as shown in FIGS. 3-4 can be used in combination with prosthetic device 100 and/or prosthetic device 400 described herein. In some examples, over-expansion structure 488 shown in FIGS. 5A-5B or the over-expansion structure 588 shown in FIGS. 6A-6B can be used in combination with the prosthetic device 100 shown in FIGS. 1A-1B.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Prosthetic devices disclosed herein can be advanced through a patient's vasculature on delivery apparatuses. The prosthetic devices can include one or more expansion and locking mechanisms that can be actuated using the delivery apparatuses to radially expand the prosthetic device and lock the prosthetic devices in one or more radially expanded states. As one example, the prosthetic devices can be crimped on or retained by the delivery apparatuses in a radially compressed state during delivery, and then radially expanded (and axially shortened) to a radially expanded state once the prosthetic devices reach the implantation site. It is understood that the prosthetic devices disclosed herein may be used with a variety of implant delivery apparatuses, and examples thereof will be discussed in more detail later.

FIGS. 1A-1B illustrate an exemplary prosthetic device (for example, a prosthetic heart valve) that can be advanced through a patient's vasculature, such as to a native heart valve, by a delivery apparatus, such as the exemplary delivery apparatus shown in FIG. 2. The frame of the prosthetic device can include mechanical expansion and locking mechanisms that can be integrated into the frame-specifically, into axially extending posts of the frame. The mechanical expansion and/or locking mechanisms can be removably coupled to, and/or actuated by, the delivery apparatus. Specifically, the mechanical expansion and/or locking mechanisms can be removably coupled to an actuator assembly (FIGS. 3-4) of the delivery apparatus, which in turn can be actuated by a physician by adjusting and/or manipulating one or more input devices (for example, one or more knobs, buttons, drawstrings, etc.) that can be included on a handle of the delivery apparatus.

As illustrated in FIGS. 5A-5B and FIGS. 6A-6B, the frame of the prosthetic device can include over-expansion structures that are configured to permit extra radial expansion of the prosthetic device (beyond that which can be provided by the mechanical expansion and locking mechanisms). Specifically, although the mechanical expansion and locking mechanisms are configured to lock the frame in a radially expanded state, the aforementioned over-expansion structures on the frame are configured to selectively disengage/unlock the frame from the expansion and locking mechanisms to permit the frame to further radially expand. For example, the over-expansion structures can be configured to deflect when a sufficiently large force is provided to the frame to permit the frame to move relative to the expansion and locking mechanisms, thereby allowing the frame to further radially expand. This over-expansion of the prosthetic device can be desirable, for example, during a valve-in-valve (“ViV”) procedure where a second prosthetic valve (which can be referred to as a guest valve) is radially expanded within the first, previously implanted prosthetic valve (which can be referred to as a host valve). The guest prosthetic valve (or other type of implantable prosthetic device) can push the host prosthetic valve (or other type of implantable prosthetic device) radially outward with sufficient force to cause the over-expansion structures in the host prosthetic valve to deflect and allow for further radially expansion of the host prosthetic valve. This may be desirable, for example, to provide more room for the guest prosthetic valve and/or to provide a tighter seal with the native tissue.

### Examples of the Disclosed Technology

FIGS. 1A-1B illustrate an exemplary example of a prosthetic device 100 (which also may be referred to herein as “prosthetic heart valve 100” and/or “prosthetic valve 100”) having a frame 102. FIG. 1B shows the frame 102 by itself, while FIG. 1A shows the frame 102 with an optional valvular structure 150 (which can comprise leaflets 158, described further below) and an optional skirt assembly. While only one side of the frame 102 is depicted in FIG. 1B, it should be appreciated that the frame 102 forms an annular structure having an opposite side that is substantially identical to the portion shown in FIG. 1B.

The frame 102 comprises an inflow end 108, an outflow end 110, and a plurality of axially extending posts 104 extending therebetween. Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame, as further described below. One or more of posts 104 can be configured as support posts 107. The actuator mechanisms 106 (which can be used to radially expand and/or radially compress the prosthetic device 100) can be integrated into the frame 102 of the prosthetic device 100, thereby reducing the crimp profile and/or bulk of the prosthetic device 100. Integrating the expansion and locking mechanisms 106 into the frame 102 can also simplify the design of the prosthetic device 100, making the prosthetic device 100 cheaper and/or easier to manufacture. In the illustrated example, an actuator 126 extends through each pair of axially aligned posts 122, 124. In other examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic device 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic device 100 can include different numbers of support posts 107 and pairs of posts 122, 124 and/or the pairs of posts 122, 124 and the support posts 107 can be arranged in a non-alternating order, in other examples.

As illustrated in FIG. 1B, the struts 112 can include a first row of struts 113 at or near the inflow end 108 of the prosthetic device 100, a second row of struts 114 at or near the outflow end 110 of the prosthetic device 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the posts 107. Each second cell 118 can be formed by two struts 115a, 115b of the third row of struts 115 and two struts 116a, 116b of the fourth row of struts 116. As illustrated in FIG. 1B, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline of the frame 102 than the struts 113a-114b).

As illustrated in FIG. 1B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and thirteen posts 104. However, in other examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame and are axially separated from one another by a gap G (FIG. 1B) (those with actuators 126 can be referred to as actuator posts or actuator struts). Each first post 122 (that is, the lower post shown in FIG. 1B) can extend axially from the inflow end 108 of the prosthetic device 100 toward the second post 124, and the second post 124 (that is, the upper post shown in FIG. 1B) can extend axially from the outflow end 110 of the prosthetic device 100 toward the first post 122. For example, each first post 122 can be connected to and extend from an inflow apex 119a and each second post 124 can be connected to and extend from an outflow apex 119b. Each first post 122 can include an inner bore 125a and each second post 124 can include an inner bore 125b. The bores 125a, 125b of each pair of actuator posts 122, 124 can receive an actuator member, such as in the form of a substantially straight threaded rod or bolt 126 as shown in the illustrated example. The threaded rod 126 also may be referred to herein as actuator 126, actuator member 126, and/or screw actuator 126. In examples where the delivery apparatus can be releasably connected to the outflow end of the frame, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore 125b of the second post (through which a rod 126 extends) can have a smooth and/or non-threaded inner surface to allow the rod 126 to slide freely within the bore 125b. Rotation of the threaded rod 126 relative to the nut produces radial expansion and compression of the frame 102, as further described below.

In some examples, the threaded rod 126 can extend past the nut 127 toward the inflow end of the frame into the inner bore 125a of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the post 122 unless the threaded rod 126 is rotated relative to the nut 127. Thus, the threaded rod 126 can be retained or held by the nut 127 and can only be moved relative to the nut 127 and/or the post 122 by rotating the threaded rod 126 relative to the nut 127 and/or the post 122. In other examples, in lieu of using the nut 127, at least a portion of the inner bore 125a of the first post 122 can be threaded. For example, the bore 125a along the end portion 128 of the first post 122 can comprise inner threads that engage the external threaded rod 126 such that rotation of the threaded rod causes the rod 126 to move axially relative to the first post 122.

When a threaded rod 126 extends through and/or is otherwise coupled to a pair of axially aligned posts 122, 124, the pair of axially aligned posts and the threaded rod can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic device 100 can include six pairs of posts 122, 124, and each of the six pairs of posts 122, 124 with their corresponding rods 126 can be configured as one of the expansion and locking mechanisms 106 for a total of six expansion and locking mechanisms 106. In other examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic device 100). Specifically, when the threaded rod 126 is rotated relative to the nut 127, the first post 122, and the second post 124, the first and second posts 122, 124 can move axially relative to one another, thereby widening or narrowing the gap G (FIG. 1B) separating the posts 122, 124, and thereby radially compressing or radially expanding the prosthetic device 100, respectively. Thus, the gap G (FIG. 1B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, as will be described in greater detail below with reference to FIGS. 3-4, the head portion 131 can removably or releasably couple the threaded rod 126 to a respective actuator assembly of a delivery apparatus that can be used to radially expand and/or radially compress the prosthetic device 100. Second, the head portion 131 can prevent the second post 124 from moving proximally relative to the threaded rod 126 and can apply a distally directed force to the second post 124, such as when radially expanding the prosthetic device 100. Specifically, the head portion 131 can have a width greater than a diameter of the inner bore 125b of the second post 124 such that the head portion 131 is prevented from moving into the inner bore 125b of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic device 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second members 122, 124 toward one another (as shown by arrows 129 in FIG. 1B), thereby radially expanding the frame 102, while rotation of the threaded rod 126 in an opposite second direction (for example, counterclockwise) causes corresponding axial movement of the first and second members 122, 124 away from one another (as shown by arrows 130 in FIG. 1B), thereby radially compressing the frame. When the rod is rotated in the first direction, the head portion 131 of the rod 126 bears against an adjacent surface of the frame (for example, an outflow apex 119b), while the nut 127 and the first post 122 travel proximally along the rod toward the second post 124, thereby radially expanding the frame. As the frame 102 moves from a compressed configuration to an expanded configuration, the gap G between the first and second posts 122, 124 can narrow.

When the rod 126 is rotated in the second direction, the rod 126 and the stopper 132 move toward the outflow end 110 of the frame until the stopper 132 abuts the inflow end 170 of the second post 124. Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic device 100.

Thus, each of the second posts 124 can slide axially relative to the second post 124 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic device 100 while the stopper 132 can apply a proximally directed force to the second post 124 to radially compress the prosthetic device 100. As explained above, radially expanding the prosthetic device 100 axially foreshortens the prosthetic device 100, causing an inflow end portion 134 and outflow end portion 136 of the prosthetic device 100 to move towards one another axially, while radially compressing the prosthetic device 100 axially elongates the prosthetic device 100, causing the inflow and outflow end portions 134, 136 to move away from one another axially.

In other examples, the rod 126 can be fixed against axial movement relative to the second post 124 (and the stopper 132 can be omitted) such that rotation of the rod 126 in the first direction produces proximal movement of the nut and radial expansion of the frame and rotation of the rod in the second direction produces distal movement of the nut and radial compression of the frame.

As also introduced above, some of the posts 104 can be configured as support posts 107. As shown in FIG. 1B, the support posts 107 can extend axially between the inflow and outflow ends 108, 110 of the frame 102 and each can have inflow end portion 138 and outflow end portion 139. The outflow end portion 139 of one or more support posts 107 can include a commissure support structure or member 140. The commissure support member 140 can comprise first and second commissure arms 142, 144 defining a commissure opening 146 between them. The outflow end of each commissure arm 142, 144 can include a tooth 148 extending into the commissure opening 146.

The commissure opening 146 can extend radially through a thickness of the post 107 and can be configured to accept a portion of a valvular structure 150 (for example, a commissure 152) to couple the valvular structure 150 to the frame 102. For example, each commissure 152 can be mounted to a respective commissure support member 140, such as by inserting a pair of commissure tabs of adjacent leaflets through the opening 146 and suturing the commissure tabs to each other and/or the arms 142, 144. In some examples, the opening 146 can be fully enclosed by the post 107 (for example, not extending to the outflow edge) such that a portion of the valvular structure 150 can be slid radially (rather than axially) into the commissure opening 146 during assembly. The teeth 148 can help retain the commissure 152 within the commissure opening 146. In the illustrated example, the commissure opening 146 has a substantially rectangular shape and extends to the distal end of the post 104. However, in other examples, the commissure opening can have any of various shapes (for example, square, oval, square-oval, triangular, L-shaped, T-shaped, C-shaped, etc.).

Though only one support post 107 comprising a commissure support member 140 is shown in FIG. 1B, it should be noted that the frame 102 can comprise any number of support posts 107, any number of which can be configured as commissure support members 140. For example, the frame 102 can comprise six support posts 107, three of which are configured as commissure support members 140. However, in other examples, the frame 102 can comprise more or less than six support posts 107 and/or more or less than three commissure support members 140.

The inflow end portion 138 of each support post 107 can comprise an extension 154 that extends toward the inflow end 108 of the frame 102. Each extension 154 can comprise an aperture 156 extending radially through a thickness of the extension 154. In some examples, the extension 154 can extend such that an inflow edge of the extension 154 aligns with or substantially aligns with the inflow end of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts can be coupled. For example, sutures used to connect the inner and/or outer skirts can be wrapped around the extensions 154 and/or can extend through openings 156.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the proximal posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic device 100 can be placed in a radially compressed state along the distal end portion of a delivery apparatus for insertion into a patient's body. When at the desired implantation site, the frame 102 (and therefore the prosthetic device 100) can be radially expanded from the radially compressed state to a radially expanded state via actuation of actuation of actuation assemblies of the delivery apparatus (as further described below), which rotate the rods 126 to produce expansion of the frame. During delivery to the implantation site, the prosthetic device can be placed inside of a delivery capsule (sheath) to protect against the prosthetic device contacting the patient's vasculature, such as when the prosthetic device is advanced through a femoral artery. The capsule can also retain the prosthetic device in a compressed state having a slightly smaller diameter and crimp profile than may be otherwise possible without a capsule by preventing any recoil (expansion) of the frame once it is crimped onto the delivery apparatus.

In other examples, the frame 102 can be formed from a self-expandable material (for example, Nitinol). When the frame is formed from a self-expandable material, the prosthetic device can be radially compressed and placed inside the capsule of the delivery apparatus to maintain the prosthetic device in the radially compressed state while it is being delivered to the implantation site. When at the desired implantation site, the prosthetic device is deployed or released from the capsule. In some examples, the frame (and therefore the prosthetic device) can partially self-expand from the radially compressed state to a partially radially expanded state. The frame 102 (and therefore the prosthetic device 100) can be further radially expanded from the partially expanded state to a further radially expanded state via actuation of actuation of actuation assemblies of the delivery apparatus (as further described below), which rotate the rods 126 to produce expansion of the frame.

As illustrated in FIG. 1A, the prosthetic valve 100 can further include the valvular structure 150, which is coupled to and supported inside the frame 102. The valvular structure 150 is configured to regulate the flow of blood through the prosthetic valve 100, from the inflow end to the outflow end. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form the commissures 152, each of which can be secured to a respective commissure support member 140 and/or to other portions of the frame 102.

In the example depicted in FIG. 1A, the valvular structure 150 includes three leaflets 158, which can be arranged to collapse in a tricuspid arrangement. Each leaflet 158 can have an inflow or cusp edge portion 160. As shown in FIG. 1A, the inflow edge portions 160 of the leaflets 158 can define an undulating, curved scallop edge that generally follows or tracks portions of the struts 112 of frame 102 in a circumferential direction when the frame 102 is in the radially expanded configuration. The inflow edges 160 of the leaflets can be referred to as a “scallop line.”

As shown in FIG. 1A, the inflow edge portions 160 of the leaflets 158 can be sutured to an inner skirt 164 generally along the scallop line. The inner skirt 164 can in turn be sutured, via one or more sutures 162, for example, to adjacent struts 112 of the frame 102. In other examples, the leaflets 158 can be sutured directly to the frame 102 along the scallop line.

The prosthetic valve 100 can further include one or more skirts or sealing members. For example, the prosthetic valve 100 can include the inner skirt 164, mounted on the radially inner surface of the frame 102. The inner skirt 164 can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic device 100.

The prosthetic device 100 can further include an outer skirt 166 mounted on the outer surface of the frame 102. The outer skirt 166 can be secured to the frame, such as with sutures 168 extending through the skirt 166 and around selected struts 112 of the frame. As noted above, the inflow edge portion of the outer skirt 166 optionally can be secured to the extensions 154, such as with sutures that extend through the apertures 156 and the skirt 166. The outer skirt 166 can function as a sealing member for the prosthetic device 100 by sealing against the tissue of the native valve annulus and helping to reduce paravalvular leakage past the prosthetic device 100.

The inner and outer skirts 164, 166 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Patent Application Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

As introduced above, the threaded rods 126 can removably couple the prosthetic device 100 to actuator assemblies of a delivery apparatus. Referring to FIG. 2, it illustrates an exemplary delivery apparatus 200 for delivering a prosthetic device or valve 202 (for example, prosthetic device 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the outer shaft 206, a second elongated shaft 209 (which comprises an inner shaft in the illustrated example) extending through the first shaft 206, and a nosecone 210 coupled to a distal end portion of the second shaft 209. The inner shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 210, 212, 214 included on the handle 204 of the delivery apparatus 200.

Though the illustrated example shows two actuator assemblies 208 for purposes of illustration, it should be understood that one actuator 208 can be provided for each actuator (for example, actuator 126) on the prosthetic valve. For example, three actuator assemblies 208 can be provided for a prosthetic valve having three actuators. In other examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the outer shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the outer shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counter-clockwise) can advance the sheath 216 distally. In other examples, the first knob 211 can be actuated by sliding or moving the knob 211 axially, such as pulling and/or pushing the knob. In other examples, actuation of the first knob 211 (rotation or sliding movement of the knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208, as will be described in greater detail below with reference to FIGS. 3-4. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In other examples, the second knob 212 can be actuated by sliding or moving the knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod), as further described below. Once the actuator assemblies 208 are uncoupled from the prosthetic valve 202, the delivery apparatus 200 can be removed from the patient, leaving just the prosthetic valve 202 in the patient.

Referring to FIGS. 3-4, they illustrate how each of the threaded rods 126 of the prosthetic device 100 can be removably coupled to an exemplary actuator assembly 300 (for example, actuator assemblies 208) of a delivery apparatus (for example, delivery apparatus 200). Specifically, FIG. 4 illustrates how one of the threaded rods 126 can be coupled to an actuator assembly 300, while FIG. 3 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 1B). The head portion 131 can comprise first and second protrusions 182 defining a channel or slot 184 between them, and one or more shoulders 186. As discussed above, the head portion 131 can have a width greater than a diameter of the inner bore 125b (FIG. 1B) of the second post 124 (FIG. 1B) such that the head portion 131 is prevented from moving into the inner bore 125b of the second post 124 and such that the head portion 131 abuts the outflow end 110 of the frame 102. In particular, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 3-4 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this example can include the same features described previously for delivery apparatus 200. In particular examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In other examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the second actuation member 304. As shown in FIGS. 3-4, the elongated elements 308 can be configured to be biased radially outward to an expanded state, for example, by shape setting the elements 308.

As shown in FIG. 4, to couple the actuator assembly 300 to the threaded rod 126, the driver 304 can be positioned such that the central protrusion 306 is disposed within the slot 184 (FIG. 3) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (that is, over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic device. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated example the central protrusion 306 has a substantially rectangular shape in cross-section, in other examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304. As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

Referring to FIGS. 5A-5B, they illustrate an over-expansion structure that can be included in the frame of the prosthetic device to allow the prosthetic device to further radially expand (beyond what is permitted by the expansion and locking mechanisms) when desired, such as when radially expanding a second prosthetic device within the prosthetic device during a valve-in-valve (ViV) procedure or when it is desired to further expand the prosthetic device after it is implanted. Although FIGS. 5A-5B illustrate an outflow end portion 436 of a frame 402 of a prosthetic device 400, it should be appreciated that the prosthetic device 400 can be the same as and/or similar to prosthetic device 100 and/or prosthetic valve 202 and can include all of the components previously described for the prosthetic devices 100, 202 with the addition of an over-expansion structure 488.

Further, the over-expansion structures 488 illustrated in FIGS. 5A-5B can be included in the frame 102 of the prosthetic device 100. Thus, the prosthetic device 400 can be a prosthetic heart valve that includes a leaflet assembly and other components described above. Components of the prosthetic device 400 that are the same as and/or similar to components of the prosthetic device 100 are similarly numbered for convenience. For example, frame 402, struts 414a,b, 416a,b, second posts 424, threaded rod 426, and head portion 431 of the threaded rod 426 can correspond to frame 102, struts 114a,b, 116a,b, second posts 124, threaded rod 126, and head portion 131. For conciseness, these similarly numbered components may not be re-introduced or otherwise discussed again in the description of FIGS. 5A-5B herein.

FIG. 5A illustrates the prosthetic device 400 in a first radially expanded state and FIG. 5B illustrated the prosthetic device 400 in a second radially expanded state (for example, over-expanded state) that is an even more radially expanded state than the first radially expanded state. For example, the prosthetic device 400 can be expanded to the first radially expanded state (FIG. 5A) when it is implanted in the native anatomy and the prosthetic device 400 can operate for its lifetime (for example, years) at this functional diameter. Then, when another prosthetic device (for example, a new prosthetic valve) is radially expanded inside the prosthetic device 400 (such as when the prosthetic device 400 has reached the end of its life), the prosthetic device 400 can be further radially expanded to the over-expanded state shown in FIG. 5B to accommodate the second prosthetic device and/or to provide an enhanced seal with the native anatomy to reduce leakage. Alternatively, it may be desired to over-expand the prosthetic device before it has reached its useful life. For example, it may be desired to over-expand the prosthetic device after the initial implantation if the native annulus in which the prosthetic device is implanted enlarges, such as from growth of the patient. The prosthetic device 400 can be overexpanded, for example, using a balloon of a balloon catheter that is inflated inside of the prosthetic device 400 to produce such overexpansion.

The over-expansion structure 488 can permit this radial over-expansion. Specifically, as explained above, the threaded rods 426 can lock the prosthetic device 400 at a particular valve diameter and/or radially expanded state. The over-expansion structure 488 can allow the frame 402 to further expand, even when the frame 402 is otherwise locked by the expansion and locking mechanisms 406. Specifically, the over-expansion structure 488 can allow the frame 402 to move relative to the threaded rod 426 to permit additional radial expansion of the frame 402 even when the expansion and locking mechanisms 406 remain idle. Thus, the over-expansion structure 488 can allow for the prosthetic device 400 to be further radially expanded without actuating the threaded rods 126. In this way, the over-expansion structure 488 can serve as another expansion mechanism that is separate from the expansion and locking mechanism 406. That said, the over-expansion structure 488 can be selectively engaged only in limited circumstances, such as when a second valve is radially expanded within the prosthetic valve 400 during a valve-in-valve (ViV) procedure or when the prosthetic valve 400 is over-expanded during its useful life using a balloon or another expansion mechanism. Thus, the expansion and locking mechanisms 406 can be the primary expansion mechanism that is used under most circumstances to radially expand the prosthetic device 400, while the over-expansion structure 488 can be utilized in very limited circumstances to provide some additional radial expansion.

The over-expansion structure 488 can be integrally formed with the frame 402 and can include flexible arms or members 490 that can extend distally from a distal end of the second post 424 and that can define an over-expansion cavity 492 at the distal end of the second post 424. In some examples, each over-expansion structure 488 can include two flexible members 490. However, in other examples, each over-expansion structure 488 can include more than two flexible members 490. In some examples, the over-expansion cavity 492 can be at least 1/20, at least 1/15, at least 1/10, at least ⅛, at least ¼, at least ⅓, at least ½, at most ¾, at most ⅔, at most ½, at most ⅓, at most ¼, and/or at most ⅙, of the length of the proximal post 424 (distance between the proximal and distal ends of the proximal post 424). Each second post 424 of the frame that includes a threaded rod 426 desirably includes an over-expansion structure 488.

The flexible members 490 can include hooked ends 494 that extend inwardly from the rest of the flexible members 490, towards the threaded rod 426. In some examples, the hooked ends 494 can directly contact the threaded rod 426. In other examples, the hooked ends 494 can be spaced away from the threaded rod 426 such that they do not frictionally engage the threaded rod 426. Regardless of whether or not they contact the threaded rod 426, the hooked ends 494 can define an opening that is smaller than the stopper 432, thereby keeping the stopper 432 outside of the cavity 492, as shown in FIGS. 5A-5B. Thus, the flexible members 490 can be naturally biased to a closed position/state, such as the closed position shown in FIGS. 5A-5B, in which they prevent the stopper 432 from entering the cavity 492.

However, when a sufficient radially outward expansion force is provided to the frame 402, the expansion of the frame applies a proximally directed axial force to the rod 426 such that the stopper 432 can push the flexible members 490 aside (to an open position/state) and enter the cavity 492. That is, the flexible members 490 can be flexible enough to deflect outwardly towards the open position/state, away from the threaded rod 126 (as shown by arrows 495 in FIG. 5A), to permit the stopper 432 to move inside the cavity 492 when the sufficient expansion force is supplied to the frame 402. For example, this expansion force can be provided to the frame 402 when a second prosthetic device is radially expanded within the prosthetic device 400. Specifically, as the second prosthetic device is radially expanded within the prosthetic device 400 (for example, by an inflatable balloon), the second prosthetic device can push radially outwards on the prosthetic device 400. As the frame is overexpanded, the axial force applied by the stopper 432 to the flexible members 490 exceeds a threshold force, and the stopper 432 can overcome the resistance of the flexible members and enter the cavity 492. In some examples, the threshold pressure from a balloon expanding the second prosthetic device or dilating the first prosthetic device required to overcome the flexible members 490 is approximately 7 Bar. However, in other examples, this threshold pressure is at least 3 Bar, at least 5 Bar, at least 7 Bar, at least 9 Bar, at least 11 Bar, at least 15 Bar, at least 20 Bar, at most 100 Bar, at most 50 Bar, at most 25 Bar, at most 20 Bar, at most 15 Bar, and/or at most 10 Bar.

Since the stopper 432 is fixedly coupled to the threaded rod 426, the entire threaded rod 426, including the stopper 432, can move proximally relative to the second post 424 of the frame 402 during the over-expansion, as shown in FIGS. 5A-5B. Stated another way, the second post 424 of the frame 402 can move distally relative to the threaded rod 426, towards a corresponding first post (for example, a first post 122), during the over-expansion. This relative movement between the threaded rod 426 and the second post 424 can be readily seen in FIGS. 5A-5B via the movement of the head portion 431 of the threaded rod 426. Specifically, in FIG. 5A, the head portion 431 can be fully received within a recess 441 at the proximal end of the second post 424 of the frame 402, while in FIG. 5B, the head portion 431 of the threaded rod 426 protrudes out of the recess 441, past the outflow end of the frame 402. The recess 441 can be configured to receive the head portion 431 of the threaded rod when the prosthetic device 400 is not radially over-expanded. In such examples, the head portion 431 of the threaded rod 426 can fit inside, engage, and/or contact the walls of the recess 441 of the second post 424 when the prosthetic device 400 is not radially over-expanded.

As the stopper moves proximally up inside the cavity 492 in FIG. 5B during the radial over-expansion process, the entire threaded rod 426 including the head portion 431 can also move proximally relative to the second post 424. As introduced above, the axial movement of the second post 424 relative to the threaded rod 426 allows the second post 424 and the first post to move towards one another, axially foreshortening and thereby radially expanding the prosthetic device 400. As the prosthetic device 400 radially over-expands, its diameter increases, as can be seen by the struts 414a,b deflecting circumferentially outwards in FIG. 5B relative to FIG. 5A.

After the stopper 432 clears the hooked ends 494 of the flexible members 490 and enters the cavity 492, the flexible members 490 can deflect inwards towards the threaded rod 426, back to their initial, rest position shown in FIGS. 5A-5B. Thus, the flexible members 490 can be formed such that they are naturally biased to the closed position/state shown in FIGS. 5A-5B. However, while the flexible members 490 can be configured to be flexible enough to deflect away from the threaded rod 426 when a sufficient radial outwardly directed expansion force is applied to the frame 402 and the axial force of the stopper 432 against the flexible members 490 exceeds a threshold force, such as during a valve-in-valve procedure, the flexible members 490 can be stiff/rigid enough to not deflect away from the rod 426 otherwise, such as when the stopper bears against ends 494 during crimping of the prosthetic device 400. For example, the flexible members 490 can be stiff enough such that during crimping of the prosthetic device 400, the flexible members 490 do not deflect away from the rod 426 and do not permit the stopper 432 to enter the cavity 492. Thus, the flexible members 490 may not deflect away from the rod 426 when less than the threshold force is applied by the stopper 432 to the flexible members during crimping and may only deflect away from the rod 426 when at least the threshold axially force is applied by the stopper to the flexible members during overexpansion. When the rod 426 is rotated to crimp the prosthetic device, the stopper 432 applies a proximally directed force to the flexible members 490 (which do not deflect and allow movement of the stopper into the cavity 492), causing the second post 424 to move away from its corresponding first post, thereby radially compressing the prosthetic device.

In some examples, the flexible members 490 can be constructed and/or otherwise formed from the same material as the rest of the frame 402 and can be made to be as stiff and/or flexible as desired by adjusting the geometry, dimensions, thickness, etc., of the flexible members 490. In other examples, the materials of the flexible members 490 can be selected to achieve a desired stiffness and/or elasticity in addition to, or in place of, adjusting their geometry.

A prosthetic device or valve (for example, prosthetic device 100, prosthetic valve 202, prosthetic device 400) can be deployed in the following exemplary manner. Generally, the prosthetic valve can be placed in a radially compressed state (for example, crimped) at or near the distal end of the delivery apparatus (for example, delivery apparatus 200 shown in FIG. 2). As introduced above, the prosthetic device can be crimped and/or otherwise radially compressed by rotating the threaded rods (for example, threaded rods 126, 426) in the second direction (for example, counterclockwise). However, as described above with reference to FIGS. 5A-5B, in certain examples, only a certain amount (that is, less than a threshold amount) of rotational force is supplied to the threaded rods during the crimping process. As discussed above, the axial force applied by the stopper 432 to the flexible arms 490 during crimping is less than the threshold force applied by the stopper 432 to the flexible arms 490 when an outward radial expansion force is applied to the frame during over expansion. Thus, the flexible arms 490 maintain the stopper 432 outside of the over expansion cavity during the crimping process.

Once the prosthetic valve is crimped on the delivery apparatus, the delivery apparatus and the prosthetic valve can be advanced over a guidewire through the vasculature of a patient to a selected implantation site (for example, the native aortic annulus). For example, when implanting the prosthetic valve within the native aortic valve, the delivery apparatus and the prosthetic valve can be inserted into and through a femoral artery, and through the aorta to the native aortic valve.

Once at the selected implantation site, the physician can deploy the prosthetic valve distally out of a delivery sheath (for example, delivery sheath 216), if the valve is retained by such a sheath. For example, the physician can manipulate (for example, rotate) one of the control mechanisms (for example, knob 211) of the delivery apparatus to retract the sheath 216 relative to the prosthetic valve. However, in other examples, the prosthetic valve may not be restrained by such a delivery sheath, and the above step can be omitted.

The physician can then radially expand the prosthetic valve by manipulating (for example, rotating) one of the control mechanisms (for example, knob 212) of the delivery apparatus to rotate the drivers (for example, drivers 304) of the actuator assemblies in a first direction (for example, clockwise), which can cause corresponding rotation of the threaded rods (for example, threaded rods 126, 426) in the first direction. As discussed above, rotation of the threaded rod in the first direction can axially foreshorten and radially expand the prosthetic device. Since the threaded rods hold/lock the prosthetic device at any valve diameter, the physician can continue to radially expand the prosthetic device until a selected diameter is achieved, without having to engage a separate locking tool. Thus, the prosthetic valve can be radially expanded in a continuous manner (for example, without the stepped expansion that results from a ratcheting mechanism) and can be continuously locked at any of various diameters.

Once the prosthetic valve has been radially expanded to an extent where it contacts and/or forms a seal with the patient's native anatomy, the patient's native anatomy (for example, the native aortic annulus) may exert compressive forces against the prosthetic valve that would tend to radially compress the prosthetic valve. However, the engagement of the threaded rod with the threaded nut prevents such forces from compressing the frame, thereby ensuring that the frame remains locked in the desired radially expanded state.

If repositioning or recapture and removal of the prosthetic valve is desired, the prosthetic valve can be radially compressed (from an expanded or partially expanded configuration) by rotating the drivers (for example, drivers 304) of the actuator assemblies (for example, actuator assemblies 300), and therefore the threaded rods, in the opposite second direction (for example, counterclockwise). For example, the physician can manipulate (for example, rotate) one of the control mechanisms (for example, knob 212) of the delivery apparatus to rotate the drivers (for example, drivers 304) of the actuator assemblies (for example, actuator assemblies 300) in the second direction (for example, counterclockwise), which can cause corresponding rotation of the threaded rods in the second direction. As discussed above, rotation of the threaded rods in the second direction causes the frame to axially elongate and radially compress. Once the prosthetic valve has been recompressed it can be repositioned at the implantation site. Once repositioned, the prosthetic valve can be radially expanded again, as described previously. The prosthetic valve can be re-compressed, repositioned, and re-expanded multiple times, as needed. In some cases, the prosthetic valve can be fully compressed and “recaptured,” that is, retracted back into the delivery sheath and/or removed from the patient's body.

Once final positioning and expansion of the prosthetic valve is achieved, the actuator assemblies (for example, actuator assemblies 300) can be released/decoupled from the prosthetic valve by retracting the sleeves (for example, sleeves 302) of the actuator assemblies relative to the drivers (for example, drivers 304) to permit the drivers to disengage from the head portion (for example, head portion 131, 431) of the threaded rods (for example, rods 126, 426), as described above. At this stage, the delivery apparatus (for example, delivery apparatus 200), including all of the actuator assemblies (for example, actuator assemblies 300), can be retracted relative to the prosthetic valve and removed from the patient's body.

Referring now to FIGS. 6A-6B, they illustrate an another example of an over-expansion structure that can be included in the frame of the prosthetic device to allow the prosthetic device to further radially expand (beyond what is permitted by the expansion and locking mechanisms) when desired, such as when radially expanding a second prosthetic device within the prosthetic device during a valve-in-valve (ViV) procedure or when it is desired to further expand the prosthetic device after it is implanted. Although FIGS. 6A-6B illustrate a portion of a frame 502 of a prosthetic device 500, it should be appreciated that the prosthetic device 500 can be the same as and/or similar to prosthetic device 100 and/or prosthetic valve 202 and can include all of the components previously described for the prosthetic devices 100, 202 with the addition of an over-expansion expansion structure 588.

Further, the over-expansion structure 588 illustrated in FIGS. 6A-6B can be included in the frame 102 of the prosthetic device 100. Thus, the prosthetic device 500 can be a prosthetic heart valve that includes a leaflet assembly and other components described above. Components of the prosthetic device 500 that are the same as and/or similar to components of the prosthetic device 100 are similarly numbered for convenience. For example, frame 502, struts 516a, 516b, first post 522, second post 524, threaded rod 526, nut 527, and stopper 532 can correspond to frame 102, struts 116a, 116b, first post 122, second post 124, threaded rod 126, nut 127, and stopper 132, respectively. For conciseness, these similarly numbered components may not be re-introduced or otherwise discussed again in the description of FIGS. 6A-6B herein.

As shown, the nut 527 is disposed in a slot 534 formed in an outflow end portion 528 of the first post 522. At least one protrusion 536 extends laterally inwardly from a side wall 538 of the slot 534. In the illustrated example, two protrusions 536 extend laterally inwardly from opposing side walls 538 of the slot. In some examples, a plurality of protrusions 536 can be disposed on each of the side walls 538. A first chamber or slot section 534a is defined between one axial end of the slot and the protrusions 536 and a second chamber or slot section 534b is defined between the other axial end of the slot and the protrusions 536. The nut 527 and the slot 534 with protrusions 536 collectively form the over-expansion structure 588. Each first post 522 of the frame that includes a threaded rod 526 desirably includes an over-expansion structure 588.

The protrusions 536 are configured to retain the nut 527 within the first slot section 534a during normal expansion of the frame 502 (for example, during the initial implantation of the prosthetic device 500) (see FIG. 6A) and permit the nut 527 to slide into the second slot section 534b during over-expansion of frame 502 such as during a subsequent valve-in-valve procedure (see FIG. 6B). Explaining further, and as described above in connection with prosthetic device 100, when rotating the threaded rod 526 in a first direction to expand the frame from a radially compressed, delivery state, a head portion of the rod 526 (for example, the head portion 431) applies a distally directed force on an adjacent surface of the second post 524 while the nut 527 travels axially relative to the rod 526 and applies a proximally directed force on the first post 522, thereby drawing first and second posts 522, 524 closer together and radially expanding the frame 502. During the radial expansion, the nut 527 bears against the protrusions 536, which retains the nut 527 within the first slot section 534a and prevents sliding movement of the rod 526 and the nut 527 relative to the first post 522. The axial force of the nut 527 against the protrusions 536 as a result of rotating the rod 526 to radially expand the frame from a radially compressed state to a radially expanded state is less than a predetermined threshold force.

The protrusions 536 are configured to permit the nut 527 to slide into the second slot section 534b if the force of the nut 527 (for example, proximally directed axial force) against the protrusions 536 is greater than the predetermined threshold force during an over-expansion procedure, such as during a post-implantation dilation of the prosthetic device 500 or during a valve-in-valve procedure, as depicted in FIG. 6B. In particular, if sufficient radial outward force is applied to the frame 502, the nut 527 can overcome the resistance of the protrusions 536 and slide into the second slot section 534b. The radial outward force can be from a balloon (or other expansion device) inflated inside the frame 502 or from a new prosthetic valve expanded within the prosthetic valve 500. Thus, the expansion force generated by the rod 526 during the initial implantation is less than what is exerted on the frame during a subsequent over-expansion procedure. In any case, by virtue of the nut 527 moving into the second slot section 534b, the second post 524 can slide distally relative to the rod 526 closer toward the first post 522, thereby allowing the frame 502 to further expand under the externally applied radial expansion force. Once the nut 527 resides in the second slot section 534b, the protrusions 536 can retain the nut 527 from sliding back into the first slot section 534a so as to assist in retaining the frame in the over-expanded state against, for example, external radial compression forces applied to the frame by the surrounding anatomy.

In some examples, the threshold pressure from a balloon expanding the second prosthetic device or dilating the first prosthetic device required to overcome the resistance of the protrusions 536 is approximately 7 Bar. However, in other examples, this threshold pressure is at least 3 Bar, at least 5 Bar, at least 7 Bar, at least 9 Bar, at least 11 Bar, at least 15 Bar, at least 20 Bar, at most 100 Bar, at most 50 Bar, at most 25 Bar, at most 20 Bar, at most 15 Bar, and/or at most 10 Bar.

The protrusions 536 can have various configuration and can be made from a variety of materials. In certain examples, the protrusions 536 can be made of a relatively soft and/or deformable material, such as an elastomer (for example, polyurethane). The protrusions 536 can be secured to the inner surfaces 538 of the slot, such as by an adhesive, fasteners, or by molding the protrusions to the surfaces 538. When the force of the nut 527 against the protrusions exceeds the predetermined threshold force, the nut 527 can compress, deflect upwardly, or otherwise deform the protrusions 536 so as to be able to slide proximally into the second slot section 534b.

In some examples, the protrusions 536 can be flexible members that can deflect and/or bend outwardly toward the inner surfaces 538 under the force of the nut 527 when it exceeds the predetermined threshold force. The flexible members forming the protrusions 536 can be made of metal and/or a polymer. In some examples, the flexible members can be integrally formed as part of the first post 522, such as by laser cutting the protrusions 536 along the inner surfaces 538 when the frame 502 is formed. In other examples, the flexible members can be separately formed and subsequently attached to the inner surfaces, such as by welding, fasteners, or an adhesive.

In some example, the nut 527 can be disposed in a channel formed in the first post 522. The channel can have a similar size and shape as the slot 534 except that the first post 522 does not have any side openings that expose the sides of the nut. In other words, the channel can completely surround the nut. In such examples, a single continuous protrusion can extend around the inner surface of the channel. The single continuous protrusion can be a flexible member or a deformable member, as previously described.

In the illustrated examples, the actuator assemblies of the delivery apparatus are releasably connected to proximal end portions of the rods (for example, rods 126) of the prosthetic device at the outflow end of the prosthetic device. In this configuration, the prosthetic device can be delivered with the delivery apparatus in a retrograde approach in which the prosthetic device is delivered through a femoral artery and the aorta to the native aortic valve. However, in other examples, it may be desirable to releasably connect the proximal end portions of the rods (for example, rods 126) to the actuator assemblies of the delivery apparatus at the inflow end of the prosthetic device, such as for delivering the prosthetic device to the native aortic valve in a transapical delivery approach or for delivering the prosthetic device to the native mitral valve in a trans-septal approach. In such examples, the position of the rods in the frame can be reversed from that shown in the figures. In particular, the rods can have head portions (for example, head portions 131) that extend proximally beyond inflow apices 119a, extend through non-threaded bores of the first posts 122, and can engage nuts (for example, nuts 127) that are disposed in the second posts 124.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic device, comprising a frame, the frame comprising: axially extending first and second posts that are axially movable relative to one another to permit the prosthetic device to radially expand and compress, wherein the first post comprises an inner bore; and a threaded rod extending through the inner bore of the first post and coupled to the second post, wherein the rod is configured to radially expand and radially compress the frame only when rotated but is otherwise configured to prevent radial expansion and compression of the prosthetic device; and wherein one of the first post and the second post further comprises an over-expansion structure that is configured to selectively permit the first and second posts to move axially closer together, when an external expansion force applied to the frame exceeds a predetermined threshold force, to allow the frame to radially expand even when the rod is not rotated.

Example 2. The prosthetic device of any example herein, particularly example 1, wherein the rod comprises a head portion positioned adjacent a first end of the first post and a stopper positioned adjacent a second end of the first post, wherein the stopper and the head portion are larger than the inner bore of the first post and thus axially constrain the first post relative to the rod.

Example 3. The prosthetic device of any example herein, particularly example 2, wherein the first post comprises the over-expansion structure.

Example 4. The prosthetic device of any example herein, particularly example 3, wherein the over-expansion structure comprises flexible arms that extend axially from the second end of the first post and define a cavity.

Example 5. The prosthetic device of any example herein, particularly example 4, wherein the flexible arms are naturally biased to a closed position in which the flexible arms prevent the stopper from entering the cavity, but are configured to selectively deflect away from closed position to an open position when an external expansion force applied to the frame exceeds the predetermined threshold to permit the stopper to enter the cavity and allow the first post to move axially relative to the rod.

Example 6. The prosthetic device of any example herein, particularly any one of examples 2-5, wherein the first post comprises a cavity at its proximal end that is configured to receive the head portion of the rod.

Example 7. The prosthetic device of any example herein, particularly any one of examples 1-6, wherein the second post comprises a threaded bore, and wherein the rod comprises a threaded portion that is configured to engage with the threaded bore to couple the rod to the second post.

Example 8. The prosthetic device of any example herein, particularly any one of examples 1-6, wherein the second post houses a threaded nut having internal threads, and wherein the rod comprises a threaded portion that is configured to engage with the internal threads of the nut to couple the rod to the second post.

Example 9. The prosthetic device of any example herein, particularly any one of examples 6-7, wherein the rod is configured to be rotated in a first direction to thread farther into the threaded bore or the threaded nut to axially compress and radially expand the prosthetic device, and wherein the rod is configured to be rotated in an opposite second direction to axially elongate and radially compress the prosthetic device.

Example 10. The prosthetic device of any example herein, particularly example 8, wherein the over-expansion structure comprises a slot form in the second post and at least one protrusion extending from an inner wall of the slot and defining first and second sections of the slot, wherein the nut is disposed in and slidably within the slot between the first and second sections of the slot, wherein the protrusion retains the nut in the first slot section when the rod is rotated to expand the frame from a compressed state to a first expanded state and permits the nut to slide into the second slot section to allow the frame to further expand to a second expanded state when an external expansion force applied to the frame exceeds the predetermined threshold force.

Example 11. The prosthetic device of any example herein, particularly any one of examples 1-10, wherein the frame comprises a plurality of pairs of first and second posts and a plurality of threaded rods, and wherein each first post or each second post comprises an over-expansion structure.

Example 12. The prosthetic device of any example herein, particularly any one of examples 1-11, further comprises a plurality of leaflets disposed in the frame and configured to regulate the flow of blood through the frame in one direction.

Example 13. A prosthetic heart valve comprising: a radially compressible and expandable frame; and a screw actuator that is coupled to the frame and configured to be rotated to radially expand the frame from a radially compressed state to a first radially expanded state; wherein the frame comprises an over-expansion structure that is configured to allow relative sliding movement between the screw actuator and the frame in a longitudinal direction to permit the frame to radially expand from the first radially expanded state to a second radially expanded state when an external expansion force applied to the prosthetic heart valve exceeds a predetermined force.

Example 14. The prosthetic heart valve of any example herein, particularly example 13, wherein the frame comprises a plurality of pairs of first and second axially extending posts, wherein the first and second posts of each pair are axially moveable relative to each other when the frame is radially expanded, wherein the screw actuator extends through a bore of the first post of a selected pair of posts and is coupled to the second post of the selected pair.

Example 15. The prosthetic heart valve of any example herein, particularly example 14, wherein the screw actuator comprises a stopper that is positioned between the first post and the second post of the selected pair, and wherein the first post comprises the over-expansion structure, which defines a cavity that is configured to selectively permit the stopper to move within the cavity, thereby allowing the frame to radially expand to the second radially expanded state when an external expansion force applied to the prosthetic heart valve exceeds a predetermined force.

Example 16. The prosthetic heart valve of any example herein, particularly example 15, wherein the over-expansion structure comprises flexible arms that extend axially from an end of the first post of the selected pair of posts and define the cavity.

Example 17. The prosthetic heart valve of any example herein, particularly example 16, wherein the flexible arms comprise hooked ends that extend inwardly towards the screw actuator.

Example 18. The prosthetic heart valve of any example herein, particularly example 16 or example 17, wherein the flexible arms are configured to be naturally biased towards a closed position in which the stopper is prevented from entering the cavity, and wherein the flexible arms are configured to deflect to an open position in which the stopper is permitted to enter the cavity when the external expansion force applied to the prosthetic heart valve exceeds the predetermined force.

Example 19. The prosthetic heart valve of any example herein, particularly any one of examples 13-18, wherein the screw actuator further comprises a head portion that is configured to apply a distally directed force to the first post of the selected pair of posts when rotated to radially expand the prosthetic heart valve.

Example 20. The prosthetic heart valve of any example herein, particularly example 19, wherein the head portion is positioned proximally relative to a proximal end of the first post of the selected pair of posts and is configured to apply the distally directed force to the proximal end of the first post of the selected pair of posts.

Example 21. The prosthetic heart valve of any example herein, particularly example 19 or example 20, wherein the first post of the selected pair of posts comprises a recess that is configured to receive the head portion of the screw actuator, and wherein the head portion is configured to move proximally out of the recess when the screw actuator moves relative to the first post during further expansion from the first radially expanded state to the second radially expanded state.

Example 22. The prosthetic heart valve of any example herein, particularly any one of examples 19-21, wherein the head portion is configured to be removably coupled to an actuator assembly of a delivery apparatus.

Example 23. The prosthetic heart valve of any example herein, particularly any one of examples 14-22, wherein the screw actuator is configured to slide within the bore of the first post of the selected pair of posts without rotating when the external expansion force applied to the frame exceeds the predetermined force.

Example 24. The prosthetic heart valve of any example herein, particularly any one of examples 14-23, wherein the second post of the selected pair of posts comprises a bore through which the screw actuator extends, wherein the bore of the second post comprises threads that engage with mating threads on the screw actuator to couple the screw actuator to the second post and that prevents radial compression of the prosthetic heart valve unless the screw actuator is rotated.

Example 25. The prosthetic heart valve of any example herein, particularly any one of examples 14-23, wherein the second post of the selected pair of posts houses a threaded nut that engages mating threads on the screw actuator to couple the screw actuator to the second post and that prevents radial compression of the prosthetic heart valve unless the screw actuator is rotated.

Example 26. The prosthetic heart valve of any example herein, particularly example 14, wherein the over-expansion structure comprises a slot form in the second post of the selected pair of posts, at least one protrusion extending from an inner wall of the slot and defining first and second sections of the slot, and a nut disposed in the slot, wherein the screw actuator has external threads that engage internal threads of the nut, wherein the nut is disposed in and slidably within the slot between the first and second sections of the slot, wherein the protrusion retains the nut in the first slot section when the rod is rotated to expand the frame from the radially compressed state to the first expanded state and permits the nut to slide into the second slot section to allow the frame to further expand to the second expanded state when the external expansion force applied to the frame exceeds the predetermined force.

Example 27. The prosthetic heart valve of any example herein, particularly any one of examples 13-26, further comprising a valvular structure comprising a plurality of leaflets supported inside of the frame, wherein the leaflets are configured to regulate the flow of blood through the frame in one direction.

Example 28. The prosthetic heart valve of any example herein, particularly any one of examples 14-27, wherein the frame comprises a plurality of axially extending third posts, each third post being located circumferentially between two pairs of first and second posts.

Example 29. The prosthetic heart valve of any example herein, particularly example 28 when dependent on example 27, wherein one or more of the third posts comprise commissure support structures that are configured to secure commissures of the leaflets to the frame.

Example 30. The prosthetic heart valve of any example herein, particularly any one of examples 13-29, further comprising at least one skirt coupled to the frame, wherein the at least one skirt comprises an inner skirt mounted on an inner surface of the frame and/or an outer skirt mounted on an outer surface of the frame.

Example 31. The prosthetic valve of any example herein, particularly any one of examples 13-30, wherein the external expansion force is configured to be supplied by an inflatable balloon positioned within the prosthetic heart valve and inflated to a pressure of at least 7 Bar.

Example 32. A prosthetic device comprising a frame, the frame comprising an expansion and locking mechanism comprising an axially extending post comprising a proximal member and a distal member that are axially movable relative to one another to permit the prosthetic device to radially expand and compress; and a rod configured to radially expand and/or radially compress the frame when actuated but that is configured to prevent radial expansion and radial compression of the prosthetic device when not actuated, wherein the rod comprises a head portion that is configured to provide an axially directed force in a first direction to the proximal member to radially expand the prosthetic device and a stopper that is configured to provide an axially directed force in a second direction to the proximal member to radially compress the prosthetic device; and wherein the proximal member of the post further comprises an over-expansion structure at a distal end of the proximal member that is configured to selectively permit the frame to radially expand even when the rod is not actuated.

Example 33. The prosthetic device of any example herein, particularly example 32, wherein over-expansion structure is configured to only permit the frame to radially expand when at least a threshold outward radial force is applied to the frame by an inflatable balloon and/or a second prosthetic device positioned within the prosthetic device.

Example 34. The prosthetic device of any example herein, particularly example 32 or example 33, wherein the rod is configured to be actuated to radially expand the prosthetic device by rotating the rod in a first direction and wherein the rod is configured to be actuated to radially compress the prosthetic device by rotating the rod in an opposite second direction.

Example 35. The prosthetic device of any example herein, particularly any one of examples 32-34, wherein the proximal member and/or the distal member of the post comprise an inner bore, and wherein the rod is configured to extend through the inner bore of the proximal member and/or the distal member.

Example 36. The prosthetic device of any example herein, particularly any one of examples 32-35, wherein the over-expansion structure comprises flexible arms that extend distally from a distal end of the proximal member and define a cavity.

Example 37. The prosthetic device of any example herein, particularly example 36, wherein the flexible arms are naturally biased to a closed position in which the flexible arms prevent the stopper from entering the cavity, but are configured to selectively deflect away from closed position to an open position to permit the stopper to enter the cavity and allow the proximal member to move axially relative to the rod.

Example 38. The prosthetic device of any example herein, particularly any one of examples 32-37, wherein the proximal member comprises a cavity at its proximal end that is configured to receive the head portion of the rod.

Example 39. The prosthetic device of any example herein, particularly any one of examples 32-38, wherein the distal member of the post comprises a threaded bore and/or a threaded nut, and where the rod comprises a threaded portion that is configured to engage with the threaded bore and/or the threaded nut of the distal member to couple the rod to the distal member.

Example 40. The prosthetic device of any example herein, particularly example 39, wherein the rod is configured to be rotated in a first direction to thread farther into the threaded bore and/or the threaded nut to axially compress and radially expand the prosthetic device, and wherein the rod is configured to be rotated in an opposite second direction to axially elongate and radially compress the prosthetic device.

Example 41. A prosthetic valve, comprising: a plurality of first posts extending between a proximal end and a distal end of the frame, each of the plurality of first posts comprising a proximal member and a distal member that are axially movable relative to one another to permit the prosthetic device to radially expand and compress, wherein the proximal member and/or the distal member comprise an inner bore; at least one rod extending through the inner bore of the proximal member of a selected one of the first posts and coupled to the distal member of the selected first post, wherein the rod is configured to radially expand and radially compress the frame only when rotated but is otherwise configured to prevent radial expansion and/or compression of the prosthetic device; wherein the proximal member of the selected first post further comprises an over-expansion structure at a distal end of the proximal member that is configured to selectively permit the proximal member to move axially relative to the rod, towards the distal member, to allow the frame to radially expand even when the rod is not rotated; a plurality of second posts extending between a proximal end and a distal end of the frame, one or more of the plurality of second posts comprising a commissure support structure; a plurality of struts extending between and connecting the plurality of first posts and the plurality of second posts; and a valvular structure comprising leaflets, wherein tabs of adjacent leaflets are secured to the commissure support structures to couple the valvular structure to the frame.

Example 42. The prosthetic valve of any example herein, particularly example 41, further comprising at least one skirt coupled to the frame.

Example 43. The prosthetic valve of any example herein, particularly example 42, wherein the at least one skirt comprises an inner skirt mounted on an inner side of the frame and/or an outer skirt mounted on an outer side of the frame.

Example 44. The prosthetic valve of any example herein, particularly any one of examples 41-43, wherein the rod comprises a head portion positioned proximally to a proximal end of the proximal member of the selected first post and a stopper positioned distally to a distal end of the over-expansion structure, wherein the stopper and the head portion are larger than the inner bore of the proximal member and thus axially constrain the proximal member.

Example 45. The prosthetic valve of any example herein, particularly example 44, wherein the over-expansion structure comprises flexible arms that extend distally from a distal end of the proximal member of the selected first post and define a cavity.

Example 46. The prosthetic valve of any example herein, particularly example 45, wherein the flexible arms are naturally biased to a closed position in which the flexible arms prevent the stopper from entering the cavity, but are configured to selectively deflect away from closed position to an open position to permit the stopper to enter the cavity and allow the proximal member of the selected first post to move axially relative to the rod.

Example 47. An assembly, comprising: a prosthetic device comprising a frame, the frame comprising one or more expansion and locking mechanisms, each of the expansion and locking mechanisms comprising: a post comprising a proximal member and a distal member that are axially movable relative to one another to permit the prosthetic device to radially expand and compress; and a rod configured to radially expand and/or radially compress the frame when actuated but that is configured to prevent radial expansion and/or radial compression of the prosthetic device when not actuated, wherein the rod comprises a head portion that is configured to provide an axial compressive force to the proximal member to radially expand the prosthetic device and a stopper that is configured to provide an axial expansive force to the proximal member to radially compress the prosthetic device; and wherein the proximal member of the post further comprises an over-expansion structure at a distal end of the proximal member that is configured to selectively permit the frame to radially expand even when the rod is not actuated. The assembly further comprises a delivery apparatus, comprising: one or more actuator assemblies configured to radially expand and/or compress the prosthetic device, each of the one or more actuator assemblies comprising a sleeve and a driver releasably coupled to the rod of one of the one or more expansion and locking mechanisms, each driver configured to rotate the rod to which it is coupled to radially expand and/or compress the prosthetic device; and a handle comprising one or more control mechanisms, at least one of the one or more control mechanisms configured to be actuated to rotate the driver of the one or more actuator assemblies and the rod of the prosthetic device to radially expand and/or compress the prosthetic device.

Example 48. The assembly of any example herein, particularly example 47, wherein the sleeve is configured to lock the driver in engagement with the head portion of the rod of the prosthetic device.

Example 49. The assembly of any example herein, particularly example 47 or example 48, wherein at least one of the one or more control mechanisms of the handle is configured to be actuated to move the sleeve axially relative to the driver to couple the one or more actuator assemblies to the prosthetic device and to decouple the one or more actuator assemblies from the prosthetic device.

Example 50. The assembly of any example herein, particularly any one of examples 47-49, wherein each rod is configured to be rotated in a first direction by one of the drivers to radially expand the prosthetic device and is configured to be rotated in an opposite second direction by one of the driver to radially compress the prosthetic device.

Example 51. The assembly of any example herein, particularly any one of examples 47-50, wherein the proximal member and/or the distal member of the post comprise an inner bore, and wherein the rod is configured to extend through the inner bore of the proximal member and/or the distal member.

Example 52. The assembly of any example herein, particularly any one of examples 47-51, wherein the over-expansion structure comprises flexible arms that extend distally from a distal end of the proximal member and define a cavity.

Example 53. The assembly of any example herein, particularly example 52, wherein the flexible arms are naturally biased to a closed position in which the flexible arms prevent the stopper from entering the cavity, but that are configured to selectively deflect away from closed position to an open position to permit the stopper to enter the cavity and allow the proximal member to move axially relative to the rod.

Example 54. A method of radially expanding an implanted prosthetic heart valve comprising a frame and at least one screw actuator, wherein the prosthetic heart valve is in a radially expanded state within a native heart valve, the method comprising: radially expanding the prosthetic heart valve from the radially expanded state to a further radially expanded state without actuating the screw actuator of the prosthetic heart valve by applying an external expansion force to the prosthetic heart valve.

Example 55. The method of any example herein, particularly example 54, wherein the external expansion force is applied to the implanted prosthetic heart valve by radially expanding a replacement prosthetic heart valve within the implanted prosthetic heart valve.

Example 56. The method of any example herein, particularly example 54, wherein the external expansion force is applied to the implanted prosthetic heart valve by inflating a balloon positioned within the implanted prosthetic heart valve.

Example 57. The method of any example herein, particularly example 56, further comprising positioning the balloon in an uninflated state within the implanted prosthetic heart valve, wherein a replacement prosthetic heart valve is mounted in a radially compressed state on the uninflated balloon, and wherein inflating the balloon comprises radially expanding the replacement prosthetic heart valve within the implanted prosthetic heart valve, which causes the implanted prosthetic heart valve to expand to the further radially expanded state.

Example 58. The method of any example herein, particularly example 56 or example 57, wherein balloon is inflated to a pressure of at least 7 Bar.

Example 59. The method of any example herein, particularly any of examples 54-58, wherein radially expanding the prosthetic heart valve comprises forcing a stopper of the screw actuator inside an over-expansion cavity integrally formed in a frame of the prosthetic heart valve.

Example 60. The method of any example herein, particularly any of examples 54-58, wherein radially expanding the prosthetic heart valve causes a threaded nut on the screw actuator to move longitudinally within a slot formed in a frame of the prosthetic heart valve.

Example 61. A prosthetic device, a prosthetic valve, or an assembly of any example herein, particularly any of examples 1-60, wherein the prosthetic device, the prosthetic valve, or the assembly is sterilized.

In view of the many possible examples to which the principles of the disclosed invention can be applied, it should be recognized that the illustrated examples are only preferred examples of the invention and should not be taken as limiting the scope of the invention. Rather, the scope of the invention is defined by the following claims and their equivalents.

1. A prosthetic device, comprising:
a frame comprising:
axially extending first and second posts that are axially movable relative to one another to permit the prosthetic device to radially expand and compress, wherein the first post comprises an inner bore; and
a threaded rod extending through the inner bore of the first post and coupled to the second post, wherein the rod is configured to radially expand and radially compress the frame only when rotated but is otherwise configured to prevent radial expansion and compression of the prosthetic device; and
wherein one of the first post and the second post further comprises an over-expansion structure that is configured to selectively permit the first and second posts to move axially closer together, when an external expansion force applied to the frame exceeds a predetermined threshold force, to allow the frame to radially expand even when the rod is not rotated.

2. The prosthetic device of claim 1, wherein the rod comprises a head portion positioned adjacent a first end of the first post and a stopper positioned adjacent a second end of the first post, wherein the stopper and the head portion are larger than the inner bore of the first post and thus axially constrain the first post relative to the rod.

3. The prosthetic device of claim 2, wherein the first post comprises the over-expansion structure.

4. The prosthetic device of claim 3, wherein the over-expansion structure comprises flexible arms that extend axially from the second end of the first post and define a cavity.

5. The prosthetic device of claim 4, wherein the flexible arms are naturally biased to a closed position in which the flexible arms prevent the stopper from entering the cavity, but are configured to selectively deflect away from closed position to an open position when an external expansion force applied to the frame exceeds the predetermined threshold to permit the stopper to enter the cavity and allow the first post to move axially relative to the rod.

6. The prosthetic device of claim 1, wherein the second post comprises a threaded bore, and wherein the rod comprises a threaded portion that is configured to engage with the threaded bore to couple the rod to the second post.

7. The prosthetic device of claim 1, wherein the second post houses a threaded nut having internal threads, and wherein the rod comprises a threaded portion that is configured to engage with the internal threads of the nut to couple the rod to the second post.

8. The prosthetic device of claim 7, wherein the over-expansion structure comprises a slot form in the second post and at least one protrusion extending from an inner wall of the slot and defining first and second sections of the slot, wherein the nut is disposed in and slidably within the slot between the first and second sections of the slot, wherein the protrusion retains the nut in the first slot section when the rod is rotated to expand the frame from a compressed state to a first expanded state and permits the nut to slide into the second slot section to allow the frame to further expand to a second expanded state when an external expansion force applied to the frame exceeds the predetermined threshold force.

9. The prosthetic device of claim 1, wherein the frame comprises a plurality of pairs of first and second posts and a plurality of threaded rods, and wherein each first post or each second post comprises an over-expansion structure.

10. The prosthetic device of claim 1, further comprises a plurality of leaflets disposed in the frame and configured to regulate the flow of blood through the frame in one direction.

11. A prosthetic heart valve comprising:
a radially compressible and expandable frame; and
a screw actuator that is coupled to the frame and configured to be rotated to radially expand the frame from a radially compressed state to a first radially expanded state;
wherein the frame comprises an over-expansion structure that is configured to allow relative sliding movement between the screw actuator and the frame in a longitudinal direction to permit the frame to radially expand from the first radially expanded state to a second radially expanded state when an external expansion force applied to the prosthetic heart valve exceeds a predetermined force.

12. The prosthetic heart valve of claim 11, wherein the frame comprises a plurality of pairs of first and second axially extending posts, wherein the first and second posts of each pair are axially moveable relative to each other when the frame is radially expanded, wherein the screw actuator extends through a bore of the first post of a selected pair of posts and is coupled to the second post of the selected pair.

13. The prosthetic heart valve of claim 12, wherein the screw actuator comprises a stopper that is positioned between the first post and the second post of the selected pair, and wherein the first post comprises the over-expansion structure, which defines a cavity that is configured to selectively permit the stopper to move within the cavity, thereby allowing the frame to radially expand to the second radially expanded state when an external expansion force applied to the prosthetic heart valve exceeds a predetermined force.

14. The prosthetic heart valve of claim 13, wherein the over-expansion structure comprises flexible arms that extend axially from an end of the first post of the selected pair of posts and define the cavity.

15. The prosthetic heart valve of claim 14, wherein the flexible arms comprise hooked ends that extend inwardly towards the screw actuator.

16. The prosthetic heart valve of claim 14, wherein the flexible arms are configured to be naturally biased towards a closed position in which the stopper is prevented from entering the cavity, and wherein the flexible arms are configured to deflect to an open position in which the stopper is permitted to enter the cavity when the external expansion force applied to the prosthetic heart valve exceeds the predetermined force.

17. The prosthetic heart valve of claim 11, wherein the screw actuator is configured to slide within the bore of the first post of the selected pair of posts without rotating when the external expansion force applied to the frame exceeds the predetermined force.

18. The prosthetic heart valve of claim 12, wherein the over-expansion structure comprises a slot form in the second post of the selected pair of posts, at least one protrusion extending from an inner wall of the slot and defining first and second sections of the slot, and a nut disposed in the slot, wherein the screw actuator has external threads that engage internal threads of the nut, wherein the nut is disposed in and slidably within the slot between the first and second sections of the slot, wherein the protrusion retains the nut in the first slot section when the rod is rotated to expand the frame from the radially compressed state to the first expanded state and permits the nut to slide into the second slot section to allow the frame to further expand to the second expanded state when the external expansion force applied to the frame exceeds the predetermined force.

19. The prosthetic heart valve of claim 11, further comprising a valvular structure comprising a plurality of leaflets supported inside of the frame, wherein the leaflets are configured to regulate the flow of blood through the frame in one direction.
